(19) United States
(12) Patent Application Publication
(10) Pub. No.: US 2022/0265668 A1
(43) Pub. Date: Aug. 25, 2022

(54) TREATMENT AND/OR PREVENTION OF LESIONS IN THE CENTRAL AUDITORY NERVOUS SYSTEM

(71) Applicant: SENSORION, Montpellier (FR)

(72) Inventor: Jonas DYHRFJELD-JOHNSEN, Lunel-Viel (FR)

(73) Assignee: SENSORION, Montpellier (FR)

(21) Appl. No.: 17/626,262

(22) PCT Filed: Jul. 24, 2020
(86) PCT No.: PCT/EP2020/071038

§ 371 Date: Jan. 11, 2022

Related U.S. Application Data
(60) Provisional application No. 62/878,437, filed on Jul. 25, 2019.

(30) Foreign Application Priority Data
Jul. 25, 2019 (EP) 19188274.5

(51) Int. Cl. A61K 31/538; A61P 27/16

(52) U.S. Cl. CPC A61K 31/538; A61K 9/0053; A61P 27/16

(57) ABSTRACT

The therapeutic use of azasetron or an analogue thereof for treating and/or preventing a lesion in the central auditory nervous system (CANS) in an individual. The treatment of lesions in the CANS with azasetron also prevents, inhibits, and/or reduces the loss of the central auditory neuron cells in the brainstem.

### FIELD OF INVENTION

The present invention relates to the treatment and/or the prevention of lesions in the central auditory nervous system (CANS). In particular, the invention relates to the therapeutic use of azasetron, or an analogue thereof, in the treatment and/or the prevention of lesions in the CANS.

### BACKGROUND OF INVENTION

The hearing or auditory system has been described to consist of the outer ear, the middle ear, the inner ear and the central auditory nervous system.

Whereas the inner ear is responsible for transforming sounds into signals that will get driven to the brain to be processed, the central auditory nervous system, often referred as the CANS, is responsible for many auditory processes, such as, inter alia, sound localization, sound lateralization, discrimination between speech sounds, recognition of auditory patterns.

Damage to the auditory system may occur on a day-to-day basis or acutely, and may result in numerous complications.

For example, noise exposure is known to disrupt the function of hair cells in the cochlea (inner ear), i.e., inner hair cells (IHCs) and outer hair cells (OHCs), and in particular OHCs. In the worst-case scenario, noise exposure may result in OHCs loss. In many cases, disruption or loss of hair cells may impair hearing sensitivity, and may result in elevated hearing thresholds.

Recent studies have shown that central components of the auditory pathway are affected both by sensory deprivation due to the cochlear deafferentation and acoustic overstimulation.

As a controlled, non-invasive experimental model, noise induced hearing loss (NIHL) may be accompanied by axon degeneration in central structures such as the cochlear nucleus, trapezoid body, and superior olivary complex. NIHL also significantly reduces cell density in higher auditory structures within all subdivisions of the medial geniculate body, the primary auditory cortex, and neuronal precursor cells in the hippocampus. In addition, spontaneous neuronal activity in the inferior colliculus (IC) has been shown to be affected. Evidence of an apoptotic pathway was found during the first week after impulse noise by TUNEL-staining within the auditory cortex, and the cingulated and piriform cortices.

In addition, it was shown that NIHL causes loss of neurons measured as decreased cell density, both immediately after noise exposure in the ventral cochlear nucleus (VCN), and 7 days after noise exposure in all investigated central auditory areas, i.e., the VCN, the dorsal cochlear nucleus (DCN), the central nucleus of the inferior colliculus (IC), the dorsal, ventral, and medial subdivision of the medial geniculate body, and the primary auditory cortex.

In addition to NIHL, numerous disorders such as seizures, brain trauma, stroke, bacterial or viral neuro-infection, demyelinating disorder, neurodegenerative disorder, neoplasm, brain malformation, learning disabilities, dyslexia, autism, depression, alcoholism, anorexia, schizophrenia, infantile mental retardation, attention deficit disorder and aged-related degeneration may provoke or involve some lesions across a variety of locations at any level of the CANS. Illustratively, partial seizures may cause auditory hallucinations.

It is believed that lesions in the CANS may lead to an overproduction of reactive oxygen species (ROS), reactive nitrogen species (RNS), as well as the activation of stress-activated MAPKs pathway, which may eventually result in a programmed cell death (apoptosis) process.

Therefore, pathophysiological changes of the central auditory pathway are relevant from a therapeutic perspective. Long-term effects involve a reduced speech intelligibility.

Early interventions may prevent these long-term effects, which can involve the cochlea as well as the downstream central auditory pathway.

Hence, there is a need to provide new approaches for a therapeutic and/or prophylactic treatment of lesions in the CANS, in particular, for stopping or reducing neuronal apoptosis following a trauma affecting any one of the structures of the CANS.

### SUMMARY

The invention pertains to azasetron, or an analogue thereof, for use in treating and/or preventing a lesion in the central auditory nervous system (CANS). In some embodiments, azasetron, or the analogue thereof, is in the form of a pharmaceutically acceptable salt and/or a solvate thereof. In some embodiments, azasetron is (R)-azasetron, (S)-azasetron, or a mixture thereof, preferably (R)-azasetron. In some embodiments, said pharmaceutically acceptable salt is selected from a group comprising or consisting of (R)-azasetron besylate, (R)-azasetron malate, and (R)-azasetron hydrochloride. In certain embodiments, azasetron, or the analogue thereof, is to be administered at a dose ranging from about 0.01 mg/kg of body weight to about 100 mg/kg of body weight. In some embodiments, azasetron, or the analogue thereof, is to be administered systemically, transdermally, or orally, preferably transdermally or orally. In certain embodiments, said lesion in the CANS is a lesion localized in the cochlear nucleus, the superior olive, the trapezoid body, the lateral lemniscus, the inferior colliculus, the medial geniculate body, and/or the auditory cortices. In some embodiments, said lesion in the CANS is a lesion observed in a disorder selected from a group comprising or consisting of a central auditory processing disorder, a stroke of the central auditory pathway, a seizure, a brain trauma, a bacterial or viral infection, a demyelinating disorder, a neurodegenerative disorder, a neoplasm, a brain malformation, learning disabilities, dyslexia, autism, depression, alcoholism, anorexia, schizophrenia, epilepsy, an infantile mental retardation, an attention deficit disorder, and aged-related degeneration. In certain embodiments, said central auditory processing disorder is an auditory processing deficit and/or a loss of speech recognition.

Another aspect of the invention relates to a pharmaceutical composition comprising azasetron, or an analogue thereof, and a pharmaceutically acceptable excipient for use in treating and/or preventing a lesion on the central auditory nervous system (CANS). In some embodiments, azasetron, or the analogue thereof, is in the form of a pharmaceutically acceptable salt and/or a solvate thereof.

A still further aspect of the invention pertains to a method for treating and/or preventing a lesion in the central auditory nervous system (CANS) in an individual in need thereof, comprising the administration to the said individual of a therapeutically effective amount of azasetron, or an analogue thereof. In certain embodiments, azasetron is in the form of a pharmaceutically acceptable salt and/or a solvate thereof.

In another aspect, the invention also relates to a method for preventing and/or inhibiting and/or reducing the loss of central auditory neuronal cells in an individual in need thereof, comprising the administration to the said individual of a therapeutically effective amount of azasetron or an analogue thereof. In some embodiments, said individual is susceptible to undergo or have undergone an acoustic trauma.

### DEFINITIONS

In the present invention, the following terms have the following meanings:
“About” preceding a figure means plus or less 10% of the value of said figure.
“Central auditory nervous system (CANS)” refers to the auditory neural pathway and structures receiving peripheral sensory input from the cochleae, including but not limited to the cochlear nucleus, the superior olive, the trapezoid body, the lateral lemniscus, the inferior colliculus, the medial geniculate body, and the auditory cortices. By definition, the CANS excludes the inner ear, middle ear and outer ear, and therefore anatomical structures such as, e.g., the cochlea and the vestibule.
“Lesion” refers to a region in an organ or tissue which has suffered damage through injury or disease. More particularly, “a lesion in the CANS” refers to the consequence of an injury or a disease in the central auditory system in the brainstem, the diencephalon, the mesencephalon and the cortex, which is materialized with cells undergoing metabolic and/or signaling changes, such as ROS production, RNS production, disturbance of calcium homeostasis and/or apoptosis engagement.
“Trauma” or “trauma event” refers to the event which is accountable for the onset of a lesion in the CANS.
“Treating” refers to reducing or alleviating at least one adverse effect or symptom of a disease, disorder or condition associated with a deficiency in, or absence of, an organ, tissue or cell function. A subject is successfully “treated” for the targeted pathologic disorder if, after receiving a therapeutic amount of the compound or composition of the present invention, the subject shows observable effects on one or more of the followings; relief to some extent, of one or more of the symptoms associated with the specific disorder or condition; and improvement in quality of life issues. The above parameters for assessing successful treatment and improvement in the disorder are readily measurable by routine procedures familiar to a physician.
“Preventing” refers to keeping from happening at least one adverse effect or symptom of a disease, disorder or condition associated with a deficiency in, or absence of, an organ, tissue or cell function.
“Therapeutically effective amount” refers to the level or the amount of an active agent that is aimed at, without causing significant negative or adverse side effects to the target, (1) delaying or preventing the onset of a central auditory disease, disorder, or condition, such as a lesion in the central auditory nervous system (CANS); (2) slowing down or stopping the progression, aggravation, or deterioration of one or more symptoms of the central auditory disease, disorder, or condition, such as a lesion in the CANS; (3) bringing about ameliorations of the symptoms of the central auditory disease, disorder, or condition, such as a lesion in the CANS; (4) reducing the severity or incidence of the central auditory disease, disorder, or condition, such as a lesion in the CANS; or (5) curing the central auditory disease, disorder, or condition, such as a lesion in the CANS. A therapeutically effective amount may be administered prior to the onset of the central auditory disease, disorder, or condition, such as a lesion in the CANS, for a prophylactic or preventive action. Alternatively, or additionally, the therapeutically effective amount may be administered after onset of the central auditory disease, disorder, or condition, such as a lesion in the CANS, for a therapeutic action. In one embodiment, a therapeutically effective amount of the composition is an amount that is effective in reducing at least one symptom of a central auditory disease, disorder or condition, such as a lesion in the CANS.
“Solvate” is used herein to describe a molecular complex comprising the compound of interest (azasetron, or an analogue thereof) and one or more pharmaceutically acceptable solvent molecules, for example, ethanol. The term “hydrate” is employed when said solvent is water.
“Analogue” refers broadly to the modification or substitution of one or more chemical moieties on a parent compound and may include functional derivatives, positional isomers, tautomers, zwitterions, enantiomers, diastereomers, racemates, isosteres, or stereochemical mixtures thereof.
“Pharmaceutically acceptable excipient” refers to an excipient that does not produce any adverse, allergic or other unwanted reactions when administered to an animal, preferably a human It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory offices, such as, for example, FDA Office or EMA.
“Individual” (or “subject”) is intended to refer to an animal individual, preferably a mammal individual, more preferably a human individual. Among the non-human mammal individuals of interest, one may non-limitationally cite pets, such as dogs, cats, rats, mice, rats, guinea pigs, hamsters, ferrets, rabbits, birds or amphibians; animals of economic importance such as cattle, sheep, goats, horses, monkeys. In one embodiment, a subject may be a “patient”, i.e. , a female or a male, an adult or a child, who/which is awaiting the receipt of, or is receiving medical care or was/is/will be the object of a medical procedure, or is monitored for the development of a specific disease, disorder or condition.

### DETAILED DESCRIPTION

WO2016/184900 discloses the therapeutic use of an inhibitor of calcineurin of the setron family for the treatment of hearing loss, such as, e.g., sensorineural hearing loss. WO2017/178645 discloses the use of (R)-azasetron ((+)-azasetron) for treating ear disorders. In other words, the use of compounds of the setron family has been reported to have a beneficial effect towards the hearing apparatus, mainly at the level of the ear itself.

With the initial goal of investigating the efficacy of an oral drug treatment to prevent neurodegeneration, neuronal cell death and activity changes in the central auditory pathway, the inventors focused on central consequences of peripheral acoustic overstimulation on the ventral cochlear nucleus, since this structure is immediately affected by an acoustic trauma.

Interestingly, the inventors provide experimental data showing that azasetron allows reducing the loss of neuronal cells in the ventral cochlear nucleus (VCN) and in the inferior colliculus (IC) after noise overstimulation (or acoustic trauma), as compared to placebo.

One aspect of the invention relates to azasetron, or an analogue thereof, for use in treating and/or preventing a lesion in the central auditory nervous system (CANS).

Another aspect of the invention further relates to the use of azasetron, or an analogue thereof, for the manufacture of a medicament for treating and/or preventing a lesion in the central auditory nervous system (CANS).

The invention also relates to the use of azasetron, or an analogue thereof, for treating and/or preventing a lesion in the central auditory nervous system (CANS).

The invention further relates azasetron, or an analogue thereof, for use for preventing and/or inhibiting and/or reducing the loss of central auditory neuronal cells. In some embodiments, the central auditory neuronal cells are localized in the cochlear nucleus, the superior olive, the trapezoid body, the lateral lemniscus, the inferior colliculus, the medial geniculate body, and/or the auditory cortices. In some embodiments, the central auditory neuronal cells are localized in the cochlear nucleus, in particular in the ventral cochlear nucleus (VCN), and/or in the inferior colliculus (IC).

In one aspect, the invention relates to azasetron, or an analogue thereof, for use for treating and/or preventing a central auditory processing disorder associated with a neurodegenerative disorder.

Azasetron is known as the 6-chloro-3,4-dihydro-N-(8-methyl-8-azabicyclo[3.2.1]-oct-3-yl)-2,4-dimethyl-3 -oxo-2H-1,4-benzoxazine-8-carboxamide or N-(1-azabicyclo[2.2.2] octan-8-yl)-6-chloro-4-methyl-3-oxo-1,4-benzoxazine-8-carboxamide and has been previously described in U.S. Pat. No. 4,892,872.

Azasetron is a compound of the following formula (I), wherein * stands for the (R)-enantiomer (or (+)-enantiomer), the (S)-enantiomer (or (−)-enantiomer), the racemate or a non-racemic mixture of (R)- and (S)-enantiomers (corresponding to mixtures of (+)-and (−)-enantiomers):

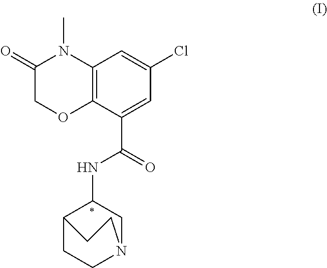 [SMILES: CN1C(=O)COc2c(C(=O)NC3CN4CCC3CC4)cc(Cl)cc21]

In some embodiments, azasetron is in the form of a pharmaceutically acceptable salt and/or a solvate thereof, and is, e.g., (R)-azasetron, (S)-azasetron, or a mixture thereof, preferably (R)-azasetron.

(R)-azasetron is a compound of the following formula (Ia), and corresponds to (+)-enantiomer:

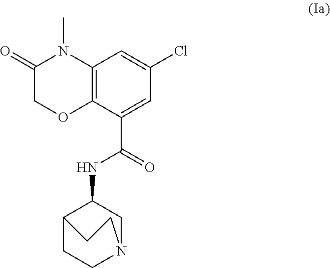 [SMILES: CN1C(=O)COc2c(C(=O)N[C@H]3CN4CCC3CC4)cc(Cl)cc21]

(S)-azasetron is a compound of the following formula (Ib), and corresponds to the (−)-enantiomer:

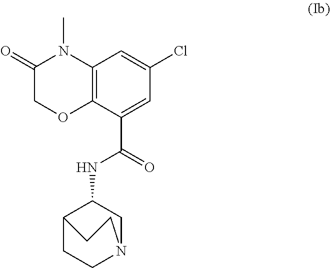 [SMILES: CN1C(=O)COc2c(C(=O)N[C@@H]3CN4CCC3CC4)cc(Cl)cc21]

In some embodiments, the azasetron, or the analogue thereof, is in the form of a pharmaceutically acceptable salt and/or a solvate thereof.

Within the scope of the instant invention, a pharmaceutically acceptable salt of azasetron (i.e., a compound of formula (I)), or an analogue thereof, includes the acid addition salt thereof. Suitable acid addition salts are formed from acids which form nontoxic salts. Examples of acid addition salts include the besylate, hydrochloride/chloride, malate, benzoate, ethane-1,2-disulfonate, fumarate, tartrate, acetate, adipate, ascorbate, aspartate, bicarbonate/carbonate, bisulphate/sulphate, borate, camsylate, citrate, cyclamate, edisylate, esylate, ethanesulfonate, formate, gluceptate, gluconate, glucuronate, glutamate, hexafluorophosphate, hibenzate, hydrobromide/bromide, hydroiodide/iodide, isethionate, lactate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, p-toluenesulfonate, tosylate, trifluoroacetate, and xinofoate salts.

In some embodiments, preferred acid addition salts include besylate, hydrochloride, malate, benzoate, ethane-1,2-disulfonate, fumarate and tartrate salts; more preferably besylate, hydrochloride, malate, and benzoate salts; even more preferably besylate, hydrochloride, or malate salts.

According to some embodiments, the invention relates to (+)-azasetron besylate, (+)-azasetron hydrochloride, (+)-azasetron malate, (+)-azasetron benzoate, (+)-azasetron ethane-1,2-disulfonate, (+)-azasetron fumarate, and/or (+)-azasetron tartrate. According to a preferred embodiment, the invention relates to (+)-azasetron besylate, (+)-azasetron hydrochloride, (+)-azasetron malate, and/or (+)-azasetron benzoate; more preferably the invention relates to (+)-azasetron besylate, (+)-azasetron hydrochloride, and/or (+)-azasetron malate.

In some embodiments, the pharmaceutically acceptable salt is selected from a group comprising or consisting of (R)-azasetron besylate, (R)-azasetron malate, and (R)-azasetron hydrochloride.

In some embodiments, azasetron is (R)-azasetron besylate.

In some embodiments, the analogue of azasetron is a benzoxazine compound of the formula (II):

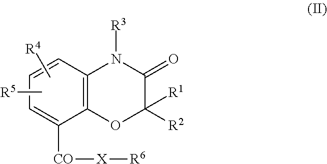

wherein R¹ and R² are the same or different, and each represents hydrogen or C₁₋₈ alkyl; R³ represents hydrogen, C₁₋₈ alkyl, phenylalkyl or substituted phenylalkyl; R⁴ and R⁵ are the same or different, and each represents hydrogen, halogen, C₁₋₈ alkyl, alkoxy, amino, acylamino, C₂₋₅ alkylamino, hydroxy or nitro; X represents oxygen or NH; R⁶ represents a group of the formula (III):

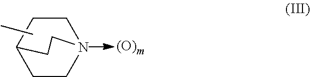

wherein m is 0 or 1,
or a group of the formula (IV):

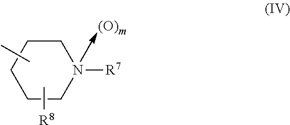

wherein R⁷ represents C₁₋₈ alkyl, phenyl C₁₋₄ alkyl, phenoxyalkyl, substituted phenyl C₁₋₄ alkyl or substituted phenoxyalkyl, R⁸ represents hydrogen or C₁₋₈ alkoxy and m is as defined above,
or a group of the formula (V):

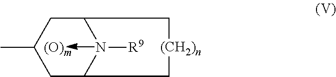

wherein R⁹ represents C₁₋₈ alkyl, phenyl C₁₋₄ alkyl or substituted phenyl C₁₋₄ alkyl,
n is 0 or 1, and m is as defined above,
or a pharmaceutically acceptable salt thereof.

In some embodiments, said azasetron analogue is selected from the group comprising or consisting of 6-chloro-3,4-dihydro-2-methyl-3-oxo-N-(3-quinuclidinyl)-2H-1,4-benzoxazine-8-carboxamide, 6-chloro-3,4-dihydro-2,4-dimethyl-3-oxo-N-(3-quinuclidinyl)-2H-benzoxazine-8-carboxamide, 6-chloro-2-ethyl-3,4-dihydro-4-methyl-3-oxo-N(3-quinuclidinyl)-2H-1,4-benzoxazine-8-carboxamide, 6-chloro-3,4-dihydro-4-methyl-3-oxo-N-(3-quinuclidinyl)-2H-1,4-benzoxazine-8-carboxamide, 6-bromo-3,4-dihydro-2,4-dimethyl-3-oxo-N-(3-quinuclidinyl)-2H-1,4-benzoxazine-8-carboxamide, and 6-chloro-3,4-dihydro-2,2,4-trimethyl-3-oxo-N-(3-quinuclidiny-1)-2H-1,4-benzoxazine-8-carboxamide, and pharmaceutically acceptable salts thereof.

Within the scope of the instant invention, the effective amount of azasetron, or an analogue thereof, to be administered may be determined by a physician or an authorized person skilled in the art and can be suitably adapted within the time course of the treatment.

In certain embodiments, the effective amount of azasetron, or an analogue thereof, to be administered may depend upon a variety of parameters, including the material selected for administration, whether the administration is in single or multiple doses, and the individual's parameters including age, physical conditions, size, weight, gender, and the severity of the disease to be treated.

An effective amount of the free-base compound of azasetron, of an analogue thereof, or a pharmaceutically acceptable salt and/or solvate thereof, preferably (+)-azasetron besylate, (+)-azasetron hydrochloride or (+)-azasetron malate may ordinarily be supplied at a dosage level from about 0.01 mg/kg to about 100 mg/kg of body weight per day, preferably from about 0.02 mg/kg to about 20 mg/kg of body weight per day, more preferably from about 0.05 mg/kg to about 5 mg/kg of body weight per day, more preferably from about 0.1 mg/kg to about 2 mg/kg of body weight per day, more preferably at about 0.5 mg/kg of body weight per day. In some embodiment, effective amounts of azasetron, or an analogue thereof, are expressed as free-base equivalent. In one embodiment, quantities of azasetron, or an analogue thereof, are expressed as salt and/or solvate equivalent.

In some embodiments, the dosage of the free-base compound of azasetron, or an analogue thereof, ranges from about 0.1 mg to about 500 mg of free-base equivalent per adult per day, preferably from about 1 mg to about 200 mg, more preferably from about 10 mg to about 100 mg, even more preferably from about 30 mg to about 60 mg of free-base equivalent per adult per day. In some embodiments, the dosage of a pharmaceutically acceptable salt and/or solvate of azasetron, or an analogue thereof, preferably (+)-azasetron besylate, (+)-azasetron hydrochloride or (+)-azasetron malate, ranges from about 0.1 mg to about 500 mg of salt and/or solvate equivalent per adult per day, preferably from about 1 mg to about 200 mg, more preferably from about 10 mg to about 100 mg, even more preferably from about 43 mg to about 87 mg of salt and/or solvate equivalent per adult per day.

In certain embodiments, azasetron, or an analogue thereof, is to be administered at a dose ranging from about 0.01 mg/kg of body weight to about 100 mg/kg of body weight.

In certain embodiments, azasetron, or an analogue thereof, is to be administered at a dose ranging from about 0.1 mg/kg of body weight to about 10 mg/kg of body weight, preferably from about 0.2 mg/kg of body weight to about 5 mg/kg of body weight.

Within the scope of the invention, “from about 0.01 mg/kg of body weight to about 100 mg/kg of body weight” includes about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95 and 100 mg/kg of body weight.

In some embodiments, azasetron, or an analogue thereof, is to be administered systemically, transdermally, or orally, preferably transdermally or orally.

In one embodiment, azasetron, or an analogue thereof, is to be administered systemically or locally.

In one embodiment, azasetron, or an analogue thereof, is to be administered by injection, orally, topically, transdermally, nasally, by inhalation, buccally, rectally, intratracheally, transmucosally, transtympanically, by percutaneous administration, intramuscularly or by parenteral administration.

In one embodiment, azasetron, or an analogue thereof, is to be administered by injection, preferably is to be systemically injected. Examples of formulations adapted to systemic injections include, but are not limited to: liquid solutions or suspensions, solid forms suitable for solution in, or suspension in, liquid prior to injection. Examples of systemic injections include, but are not limited to, intravenous, subcutaneous, intramuscular, intradermal, intravitreal, and intraperitoneal injection, or perfusion. In another embodiment, when injected, the compound, composition, pharmaceutical composition or medicament of the invention is sterile. Methods for obtaining a sterile pharmaceutical composition include, without limitation, sterile filtration, terminal sterilization (dry heat, radiation, moist heat, gases, gamma radiation) or sterilization via aseptic processing.

In some embodiments, azasetron, or an analogue thereof, is to be administered orally.

Illustratively, formulations suitable for an oral administration include, but are not limited to: solid forms, liquid forms and gels. Examples of solid forms adapted to oral administration include, but are not limited to, pill, tablet, capsule, soft gelatin capsule, hard gelatin capsule, caplet, compressed tablet, cachet, wafer, sugar-coated pill, sugar coated tablet, or dispersing and/or disintegrating tablet, powder, solid forms suitable for solution in, or suspension in, liquid prior to oral administration and effervescent tablet. Examples of liquid forms adapted to oral administration include, but are not limited to, solutions, suspensions, drinkable solutions, elixirs, sealed phial, potion, drench, syrup and liquor.

In some embodiments, azasetron, or an analogue thereof, is to be administered transdermally, preferably by the mean of a transdermal gel or a transdermal patch.

Within the scope of the instant invention, the term “transdermally” is meant to refer to the delivery of azasetron, or an analogue thereof, by passage through the skin or a mucosal tissue and into the bloodstream. In some embodiments, the term “transdermal” is equivalent to the term “percutaneous”.

In some embodiments, a patch according to the instant invention may comprise a mono or a multilayer fabric comprising an adhesive matrix, wherein at least one layer is impregnated with azasetron or an analogue thereof.

In some embodiments, the adhesive matrix comprises a silicone polymer, an acrylic polymer, polyisobutylene polymer, or a mixture thereof.

In some embodiments, the patch may comprise an impermeable layer, a backing layer, and/or a liner layer.

In some embodiments, the backing layer may comprise, but may not be limited to, synthetic polymers such as, e.g., polycarbonates, polyesters, polyethylene, poly(ethylene terephthalate), polyimides, polypropylene, polyurethanes, polyvinylchlorides, and a mixture thereof.

In some embodiments, the liner layer may comprise, but may not be limited to, metal foils; polyethylene terephthalate; polytetrafluoroethylene; cellophane; silicone-containing elastomer, film, paper or rubber; aluminum-containing paper; polyvinyl chloride film; polytetrafluoroethylene; polyether block amide copolymers; polyethylene methyl methacrylate block copolymers; polyurethanes; polyvinylidene chloride; nylon; rubber-based polyisobutylene; styrene; styrene-butadiene; styrene-isoprene copolymers; polyethylene; polypropylene and a mixture thereof.

In some embodiments, the azasetron, or an analogue thereof, is incorporated in the layer comprising an adhesive matrix.

In some embodiments, the patch according to the instant invention may comprise one or more additional ingredient(s) selected in a group comprising or consisting of a solubilizing agent, a penetration enhancing agent, a penetration delaying agent, a solvent, a surfactant and mixtures thereof.

Solubilizing agents and penetration modulating (enhancing or delaying) agents that are incorporated in transdermal formulations according to the instant invention, such as, e.g., transdermal gel or patch, may modify the pharmacokinetic parameters that define the delivery of azasetron, or an analogue thereof, into the systemic circulation via the transdermal application route.

In some embodiments, the one or more additional ingredients may be selected in a group comprising or consisting of acrylic copolymer, dimethyl acetamide, dimethyl formamide, dimethyl isosorbide, dimethyl sulfoxide, dioctyl sodium sulphosuccinate, glycerol, hexylene glycol, lauryl lactate, levulinic acid, methyl alcohol, propylene glycol, silicone oil, urea, vitamin E, and a mixture thereof.

In some embodiments, the patch according to the instant invention is latex-free and/or hypoallergenic.

A transdermal hydrogel or a transdermal patch according to the instant invention may be manufactured by any suitable method from the state of the art.

In one embodiment, azasetron, or an analogue thereof, is to be administered in an immediate-release form.

In one embodiment, azasetron, or an analogue thereof, is to be administered in a sustained-release form. In another embodiment, the composition, the pharmaceutical composition or the medicament of the invention comprises a delivery system that controls the release of azasetron, or an analogue thereof, preferably of (+)-azasetron or a pharmaceutically acceptable salt and/or solvate thereof, more preferably of (+)-azasetron besylate, (+)-azasetron hydrochloride, (+)-azasetron malate or a mixture thereof.

In another embodiment, the composition, pharmaceutical composition or medicament of the invention comprises sustained-release drug delivery agents, such as biodegradable polymers. As used herein, a sustained-release drug delivery agent is a composition, e.g., a polymeric matrix, which provides a reservoir or vehicle for release of a therapeutic agent over an extended time in a subject.

It should be appreciated that the sustained-release drug delivery agents include a variety of materials, including, but not limited to, polymeric materials that form in response to temperature change (e.g., poloxamers), polyelectrolyte complexing (e.g., chitosan/chondroitin sulfate), polymer cross-linking (both physical and chemical, e.g., with rheological synergism or hyaluronic acid derivatives, respectively), or sensitivity to photo or electromagnetic waves (e.g., UV or microwaves), solvent exchange, or pH. In certain embodiments, the sustained-release drug delivery agent is a hydrophilic material.

In the compositions of the present invention, azasetron, or an analogue thereof, alone or in combination with another active principle, can be administered as a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, gingival or mucosal or mucoadhesive formulations, aerosols, sprays, transtympanical, implants, subcutaneous, transdermal, topical, intraperitoneal, intramuscular, intravenous, subdermal, transdermal, intrathecal and intranasal administration forms, and rectal administration forms.

In some embodiments, azasetron, or an analogue thereof, may be administered once per day, twice per day or three times per day.

In some embodiments, the dosage regimen of azasetron, or an analogue thereof, may be administered for one week, two weeks, three weeks, one month, two months, three months, four months, five months, six months, or more.

In some embodiments, a lesion according to the invention may include an abscess, an arteriovenous malformation (AVM), a stroke (or cerebral infarction), a cerebral palsy, a multiple sclerosis, a brain necrosis and a brain tumor.

In some embodiments, the lesion may be localized in the brainstem, the diencephalon, the mesencephalon and/or the cortex.

As used herein, the term “brainstem” is intended to refer to the area of the brain that lies between the deep structure of the cerebral hemispheres and the cervical spinal cord.

Illustratively, the lesion may be ipsilateral, contralateral or bilateral.

In some embodiments, a lesion may result in a cell loss, in particular a loss of neurons and/or a loss of neural supporting cells, also termed neuroglia or glial cells.

Within the scope of the instant invention, the neural supporting cells encompass astrocytes, microglial cells, ependymal cells, and oligodendrocytes.

In some embodiments, the lesion in the CANS may result in an auditory processing deficit. Within the scope of the instant invention, the expression “auditory processing” encompasses both the recognition of a sound (as a signal) and the further interpretation of the sound (processing of the signal).

In practice, a lesion in the CANS may lead to a deficit and/or an impairment and/or a decreased ability of sound localization, auditory discrimination, auditory pattern recognition, speech recognition, temporal processing, processing degraded auditory signals, processing competing auditory signals.

In certain embodiments, a lesion in the CANS may result in central deafness.

Within the scope of the instant invention, the expression “central deafness” refers to “deafness” of central origin with some preserved peripheral auditory function.

In practice, central deafness may also be termed cortical deafness or generalized auditory agnosia.

In some embodiments, the central deafness may be associated with an auditory agnosia, a verbal auditory agnosia and/or an amusia.

Within the scope of the instant invention, the expression “auditory agnosia” refers to an inability to recognize sound but the preservation of spoken language recognition; the expression “verbal auditory agnosia” refers to an inability to recognize/comprehend spoken language; the expression “amusia” refers to a severe inability to appreciate auditory characteristics of music, such as melody or rhythm.

In some embodiments, a lesion in the CANS may be assessed by one or more imaging methods, one or more electrophysiologic methods and/or by one or more psychoacoustic methods, known or adapted from the state in the art.

A lesion in the CANS may be assessed by any suitable imaging method known from the state in the art. Illustratively, one may cite a computerized tomography (CT-scan), positron emission tomography (PET-scan), or magnetic resonance imaging (MRI).

A lesion in the CANS may also be assessed by any suitable electrophysiologic method known or adapted from the state in the art. Non-limitative examples of electrophysiologic methods include the Auditory Brainstem Responses (ABR), the Auditory Middle Latency Responses (AMLRs) method, the Auditory P300 Response method and Mismatch Negativity Response (MMN). For example, the ABR method comprises a step of recording the electrical activity extending from the 8^{th} cranial nerve up to the cortical auditory centers.

A lesion in the CANS may also be assessed by any suitable psychoacoustic method known or adapted from the state in the art. Non-limitative illustration of a psychoacoustic methods may include dichotic listening (e.g., different pairs of numbers presented simultaneously to each ear), interaural time perception, sound localization.

It is understood that imaging and/or electrophysiological and/or psychoacoustic methods may be employed to determine the occurrence and/or the degree of severity of a lesion in the CANS.

In some embodiments, the lesion in the CANS is a lesion localized in the cochlear nucleus, the superior olive, the trapezoid body, the lateral lemniscus, the inferior colliculus, the medial geniculate body and/or the auditory cortices. In some embodiments, the lesion in the CANS is a lesion localized in the cochlear nucleus, in particular in the ventral cochlear nucleus (VCN), and/or the inferior colliculus (IC).

As used herein, the auditory cortices encompass the primary auditory cortex lying in the superior temporal gyms of the temporal lobe and extends into the lateral sulcus and the transverse temporal gyri (also called Heschl's gyri).

Illustratively, a lesion localized in the cochlear nucleus may alter one or more neuronal cell types, such as the pyramidal cells, the spherical bushy cells, the octopus cells, the globular bushy cells and/or the multipolar cells.

A lesion may also alter neurons in the lateral superior olive (LSO) and/or the medial superior olive (MSO), which receive and compare signals from both ears upon processing by the cochlear nuclei.

A lesion in the trapezoid body may affect the sound localization process.

In some embodiments, the lesion in the CANS is a lesion observed in a disorder selected from a group comprising or consisting of central auditory processing disorder, a stroke of the central auditory pathway, a seizure, a brain trauma, a bacterial or viral infection, a demyelinating disorder, a neurodegenerative disorder, a neoplasm, a brain malformation, learning disabilities, dyslexia, autism, depression, alcoholism, anorexia, schizophrenia, epilepsy, infantile mental retardation, attention deficit disorder, and aged-related degeneration.

In some embodiments, a neurodegenerative disorder may encompass Alzheimer's disease, Parkinson's disease, Huntington's disease, amyotrophic lateral sclerosis, and Down's syndrome.

Therefore, in one embodiment, the present invention relates to azasetron, or an analogue thereof, for use in treating a central auditory processing disorder or a stroke of the central auditory pathway.

In certain embodiments, the central auditory processing disorder is an auditory processing deficit and/or a loss of speech recognition.

Within the scope of the instant invention, a “deficit and/or a loss of speech recognition” refers to the difficulty or the inability for an individual to intelligibly recognize a speech in a quiet or in a noisy environment.

Illustratively, a deficit and/or a loss of speech recognition may be assessed by the Hagerman's speech recognition test, or a test adapted therefrom.

In some embodiments, the lesion is not associated with a tinnitus, a hearing loss, in particular a sensorineural hearing loss, and/or a vestibular dysfunction.

In one embodiment, the present invention relates to the acute treatment of a lesion in the CANS.

Within the scope of the instant invention, an “acute” treatment of a lesion in the CANS refers to a treatment started as soon as possible after the onset of a lesion in the CANS, preferably at most 7 days after the onset of a lesion in the CANS.

In one embodiment, the acute treatment starts at most about 1h, about 2h, about 4h, about 6h, about 12h, about 18h, about 1 day, about 2 days, about 3 days, about 4 days, about 5 days, about 6 days or at most about 7 days after the onset of a lesion in the CANS.

In one embodiment, the present invention relates to the treatment of an acute lesion in the CANS.

Within the scope of the instant invention, an “acute lesion” refers to a lesion occurring rapidly and suddenly in an individual without known pre-existing or diagnosed lesion in the CANS.

In one embodiment, the treatment of an acute lesion in the CANS may start as soon as possible after the onset of the lesion in the CANS, preferably as soon as about 1h, about 2 h, about 3 h, about 4 h, about 5 h, about 6 h, about 12 h, about 18 h, about 24 h, about 48 hours, about 72 hours after the onset of the lesion in the CANS.

The invention also pertains to a pharmaceutical composition comprising, consisting, or consisting essentially of azasetron, an analogue thereof, or a pharmaceutically acceptable salt and/or solvate thereof and a pharmaceutically acceptable excipient.

The invention further relates to a composition comprising, consisting, or consisting essentially of azasetron, of an analogue thereof, or a pharmaceutically acceptable salt and/or solvate thereof, for use in treating and/or preventing a lesion on the central auditory nervous system (CANS).

The invention further relates to a pharmaceutical composition comprising, consisting, or consisting essentially of azasetron, of an analogue thereof, or a pharmaceutically acceptable salt and/or solvate thereof, and a pharmaceutically acceptable excipient, for use in treating and/or preventing a lesion on the central auditory nervous system (CANS).

Another object of the invention is a medicament comprising, consisting, or consisting essentially of azasetron, of an analogue thereof, or a pharmaceutically acceptable salt and/or solvate thereof, for use in treating and/or preventing a lesion on the central auditory nervous system (CANS).

In one embodiment, the composition, pharmaceutical composition or medicament of the invention comprises, consists or consists essentially of (+)-azasetron or a pharmaceutically acceptable salt and/or solvate thereof, preferably (+)-azasetron besylate, (+)-azasetron hydrochloride, or (+)-azasetron malate.

As used herein, the expression “consist essentially of”, with reference to a composition, pharmaceutical composition or medicament, means that the at least one compound of the invention is the only one therapeutic agent or agent with a biologic activity within said composition, pharmaceutical composition or medicament. The other remaining ingredients may be referred to as “excipients”.

In one embodiment, the composition, pharmaceutical composition or medicament of the invention does not comprise (−)-azasetron or a pharmaceutically acceptable salt and/or solvate thereof. In one embodiment, the composition, pharmaceutical composition or medicament of the invention comprises less than about 40% w/w, 30% w/w, 20% w/w or 10% w/w of (−)-azasetron or a pharmaceutically acceptable salt and/or solvate thereof in weight to the total weight of azasetron, preferably less than about 9%, 8%, 7%, 6%, 5%, 4%, 3%, 2%, 1% w/w (−)-azasetron or a pharmaceutically acceptable salt and/or solvate thereof in weight to the total weight of azasetron, more preferably less than about 0.5%, 0.4%, 0.3%, 0.25%, 0.2%, 0.15%, 0.1% w/w (−)-azasetron or a pharmaceutically acceptable salt and/or solvate thereof in weight to the total weight of azasetron.

In one embodiment, the composition, pharmaceutical composition or medicament of the invention does not comprise a mixture of (+)-azasetron and (−)-azasetron, or pharmaceutically acceptable salts and/or solvates thereof.

In one embodiment, the composition, pharmaceutical composition or medicament of the invention comprises at least a 60:40 w/w mixture, preferably at least a 70:30, 80:20 or 90:10 w/w mixture of (+)-azasetron:(−)-azasetron, or pharmaceutically acceptable salts and/or solvates thereof, more preferably at least a 95:5, 96:4, 97:3, 98:2, or 99:1 w/w mixture of (+)-azasetron:(−)-azasetron, or pharmaceutically acceptable salts and/or solvates thereof, even more preferably at least a 99.5:0.5, 99.6:0.4, 99.7:0.3, 99.75:0.25, 99.8:0.2, 99.85:0.15, or 99.9:0.1 w/w mixture of (+)-azasetron:(−)-azasetron, or pharmaceutically acceptable salts and/or solvates thereof.

In some embodiments, the pharmaceutically acceptable excipient may include, but may not be limited to, water, saline, Ringer's solution, dextrose solution, ion exchangers, alumina, magnesium stearate, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat, solution of ethanol, starch, glucose, sucrose, dextran, mannose, mannitol, sorbitol, polyethylene glycol (PEG), phosphate, acetate, gelatin, collagen, Carbomer, vegetable oils, the like and a mixture thereof.

In some embodiments, pharmaceutically acceptable excipients suitable for the invention may also comprise, but may not be limited to, surfactants (e.g., hydroxypropylcellulose); suitable carriers, such as, for example, solvents and dispersion media containing, for example, water, ethanol, polyol (e.g., glycerol, propylene glycol, and liquid polyethylene glycol, and the like), suitable mixtures thereof, and vegetable oils, such as, for example, peanut oil and sesame oil; isotonic agents, such as, for example, sugars or sodium chloride; coating agents, such as, for example, hydroxypropylmethylcellulose (HPMC), polyethylene glycol (PEG), polysorbate 80, titanium dioxide and lecithin; agents delaying absorption, such as, for example, aluminum monostearate and gelatin; preservatives, such as, for example, benzalkonium chloride, benzethonium chloride, chlorobutanol, thimerosal and the like; buffers, such as, for example, boric acid, sodium and potassium bicarbonate, sodium and potassium borates, sodium and potassium carbonate, sodium acetate, sodium biphosphate and the like; tonicity agents, such as, for example, dextrose, potassium chloride, propylene glycol, sodium chloride; antioxidants and stabilizers, such as, for example, sodium bisulfite, sodium metabisulfite, sodium thiosulfate, thiourea and the like; nonionic wetting or clarifying agents, such as, for example, polysorbate 80, polysorbate 20, poloxamer 282 and tyloxapol; viscosity modifying agents, such as, for example dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose; diluents, adjuvants and the like.

In some embodiments, the pharmaceutical composition further includes one or more suitable preservatives, stabilizers, antioxidants, antimicrobials, and buffering agents, such as, for example, butylated hydroxyanisole (BHA), butylated hydroxytoluene (BHT), citric acid, ascorbic acid, tetracycline, the like and mixtures thereof.

In one embodiment, the compositions contain 0.1, 0.5, 1, 10, 20, 50, 100, 250, 500, 1,000 or 2,000 mg of azasetron, of an analogue thereof, or a pharmaceutically acceptable salt and/or solvate thereof, preferably (+)-azasetron besylate, (+)-azasetron hydrochloride, or (+)-azasetron malate, for the symptomatic adjustment of the dosage to the patient to be treated. In one embodiment, quantities of azasetron or an analogue thereof, are expressed as free-base equivalent. Therefore, in one embodiment, the compositions contain 1, 5, 10, 15, 20, 25, 30, 35, 40, 45, 50, 55, 60, 65, 70, 75, 80, 85, 90, 95 or 100 mg of the free-base compound of azasetron or an analogue thereof. In one embodiment, quantities of azasetron or an analogue thereof, are expressed as salt and/or solvate equivalent. Therefore, in one embodiment, the compositions contain 1, 5, 10, 15, 20, 25, 30, 35, 40, 45, 50, 55, 60, 65, 70, 75, 80, 85, 90, 95, 100, 105, 110, 115, 120 or 125 mg of a pharmaceutically acceptable salt and/or solvate of azasetron or an analogue thereof, preferably (+)-azasetron besylate, (+)-azasetron hydrochloride or (+)-azasetron malate.

A medicament may typically contain from about 0.1 mg to about 10,000 mg of the compound of azasetron, of an analogue thereof, or a pharmaceutically acceptable salt and/or solvate thereof, preferably (+)-azasetron besylate, (+)-azasetron hydrochloride, or (+)-azasetron malate, preferably from about 0.1 mg to about 2,000 mg, more preferably from about 0.1 mg to about 500 mg, more preferably from about 0.1 mg to about 100 mg of azasetron, of an analogue thereof, or a pharmaceutically acceptable salt and/or solvate thereof, preferably (+)-azasetron besylate, (+)-azasetron hydrochloride, or (+)-azasetron malate. In one embodiment, quantities of azasetron or an analogue thereof, are expressed as free-base equivalent. Therefore, in one embodiment, the medicament contains 1, 5, 10, 15, 20, 25, 30, 35, 40, 45, 50, 55, 60, 65, 70, 75, 80, 85, 90, 95 or 100 mg of the free-base compound of azasetron or an analogue thereof. In one embodiment, quantities of azasetron or an analogue thereof, are expressed as salt and/or solvate equivalent. Therefore, in one embodiment, the medicament contains 1, 5, 10, 15, 20, 25, 30, 35, 40, 45, 50, 55, 60, 65, 70, 75, 80, 85, 90, 95, 100, 105, 110, 115, 120 or 125 mg of a pharmaceutically acceptable salt and/or solvate of azasetron or an analogue thereof, preferably (+)-azasetron besylate, (+)-azasetron hydrochloride or (+)-azasetron malate.

The invention also relates to the use of a pharmaceutical composition comprising azasetron, or an analogue thereof, and a pharmaceutically acceptable excipient, for treating and/or preventing a lesion of the central auditory nervous system (CANS).

The invention also relates to the use of a pharmaceutical composition comprising azasetron, or an analogue thereof, and a pharmaceutically acceptable excipient, for the preparation of a medicament for treating and/or preventing a lesion of the central auditory nervous system (CANS).

The invention also relates to a method for treating and/or preventing a lesion in the central auditory nervous system (CANS) in an individual in need thereof, comprising the administration to the said individual of a therapeutically effective amount of azasetron, of an analogue thereof, or a pharmaceutically acceptable salt and/or solvate thereof.

As explained above, the loss of central auditory cells, in particular central auditory neuronal cells may be the consequence of an overproduction of ROS and/or RNS, dysregulation of calcium homeostasis, excitotoxicity, ischemia and/or an apoptosis process following a trauma event affecting the central auditory central system.

Another aspect of the invention pertains to a method for preventing and/or inhibiting and/or reducing the loss of central auditory neuronal cells in an individual in need thereof, comprising the administration to the said individual of a therapeutically effective amount of azasetron or an analogue thereof. In some embodiments, the central auditory neuronal cells are localized in the cochlear nucleus, the superior olive, the trapezoid body, the lateral lemniscus, the inferior colliculus, the medial geniculate body, and/or the auditory cortices. In some embodiments, the central auditory neuronal cells are localized in the cochlear nucleus, in particular in the ventral cochlear nucleus (VCN), and/or in the inferior colliculus (IC).

In some embodiments, the invention relates to a method for increasing ability of sound localization, auditory discrimination, auditory pattern recognition, temporal processing, processing degraded auditory signals, and/or processing competing auditory signals in an individual in need thereof, comprising the administration to the said individual of a therapeutically effective amount of azasetron or an analogue thereof.

In some embodiments, the reduction of the loss of auditory neuronal cells upon administration of azasetron or an analogue thereof may reach at least about 20% and up to about 95%.

Within the scope of the invention, at least about 20% encompasses 20%, 25%, 30%, 35%, 40%, 45%, 50%, 55%, 60%, 65%, 70%, 75%, 80%, 85%, 90% and 95%.

One may assess the loss of auditory neuronal cells by determining whether the neurons are functionally maintained at the localization of interest in the CANS. In other words, one may assess the loss of auditory neuronal cells by determining the integrity of the neurons at the localization of interest in the CANS.

In practice, evaluation of the integrity of the neurons may be achieved by the means of suitable methods from the state of the art.

In some embodiments, evaluation of the integrity of the neurons may be assessed by one or more imaging methods, one or more electrophysiologic methods and/or by one or more psychoacoustic methods.

Another aspect of the invention further pertains to a method for treating and/or preventing a central auditory processing disorder, preferably associated with a stroke of the central auditory pathway, a seizure, a brain trauma, a bacterial or viral infection, a demyelinating disorder, a neurodegenerative disorder, a neoplasm, a brain malformation, learning disabilities, dyslexia, autism, depression, alcoholism, anorexia, schizophrenia, epilepsy, an infantile mental retardation, an attention deficit disorder, and/or aged-related degeneration, in an individual in need thereof, comprising the administration to the said individual of a therapeutically effective amount of azasetron, of an analogue thereof, or a pharmaceutically acceptable salt and/or solvate thereof.

In one aspect, the invention relates to a method for treating and/or preventing a central auditory processing disorder associated with a neurodegenerative disorder in an individual in need thereof, comprising the administration to the said individual of a therapeutically effective amount of azasetron, of an analogue thereof, or a pharmaceutically acceptable salt and/or solvate thereof.

The invention also relates to a method for treating a central deafness, in particular an auditory agnosia, a verbal auditory agnosia and/or an amusia in an individual having a lesion in the CANS, comprising the administration to the said individual of a therapeutically effective amount of azasetron, of an analogue thereof, or a pharmaceutically acceptable salt and/or solvate thereof.

In some further aspect, the invention relates to a method for preventing a central deafness, in particular an auditory agnosia, a verbal auditory agnosia and/or an amusia in an individual susceptible to have a lesion in the CANS, comprising the administration to the said individual of a therapeutically effective amount of azasetron, of an analogue thereof, or a pharmaceutically acceptable salt and/or solvate thereof.

In certain embodiments, the individual to be treated is not affected with a disorder of the outer ear and/or the inner ear, preferably a disorder not associated with a tinnitus, a hearing loss, in particular a sensorineural hearing loss, and/or a vestibular dysfunction.

In one embodiment, hearing loss corresponds to a decrease in hearing capacities of at least about 20, 30, 40, 50, 60, 70, 80, 90 dB or more over at least three contiguous frequencies or to total deafness. Methods for measuring hearing loss are well-known by the skilled artisan. Examples of such methods include, but are not limited to, tuning fork test, bone conduction test, pure tone audiogram, ABR (auditory brainstem responses) measurement, DPOAE (distortion product otoacoustic emissions) measurement, TEOAE (transiently evoked otoacoustic emissions) measurement, and the like.

In certain embodiments, the individual to be treated accordingly to the instant invention is susceptible to undergo or have undergone an acoustic trauma.

In some embodiments, the said acoustic trauma does not affect the outer ear and/or the middle ear and/or the inner ear.

In some embodiments, the individual is not diagnosed with a disorder of the outer ear and/or the middle ear and/or the inner ear. In certain embodiments, the individual is not diagnosed with a tinnitus, a hearing loss, in particular a sensorineural hearing loss, and/or a vestibular dysfunction.

In some embodiment, azasetron, or an analogue thereof, is administered to an individual free of any disorder of the outer ear and/or of the inner ear disorder, preferably is exclusively administered to an individual free of any disorder of the outer ear and of the inner ear.

In some embodiments, an acoustic trauma may result from being near a loud industrial equipment, being in a high-decibel environment (e.g., high-density car traffic; airport), being at a high-decibel event (e.g., concert; sport event; fireworks; military parade), being near a gun usage, being near an explosion.

In some embodiments, individuals that are susceptible to undergo an acoustic trauma may be individuals that are overexposed to noise levels over about 75 dB, preferably over about 80 dB, more preferably over about 85 dB.

In certain embodiments, a noise level over about 75 dB includes a noise level of about 75 dB, 80 dB, 85 dB, 90 dB, 95 dB, 100 dB, 105 dB, 110 dB, 115 dB, 120 dB, 125 dB or more.

In some embodiments, individuals that are susceptible to undergo an acoustic trauma may be individuals that are overexposed to noise levels of from about 75 dB to about 125 dB.

It is understood that the overexposure may depend from various parameters, such as, e.g., the intensity of the noise, as measured in dB, the frequency of the noise, since higher frequencies intend to be more damaging, the total time of exposure to the noise.

In some embodiments, the individual has a disorder selected from a group comprising or consisting of a central auditory processing disorder, a stroke of the central auditory pathway, a seizure, a brain trauma, a bacterial or viral infection, a demyelinating disorder, a neurodegenerative disorder, a neoplasm, a brain malformation, learning disabilities, dyslexia, autism, depression, alcoholism, anorexia, schizophrenia, epilepsy, an infantile mental retardation, an attention deficit disorder and aged-related degeneration.

In some embodiments, the therapeutic uses and methods provided by the instant invention may also be applied to an individual having an irremediable peripheral ear lesion.

Within the scope of the instant invention, an “irremediable peripheral ear lesion” is intended to refer to a lesion in the peripheral ear system that cannot be cured or which symptoms cannot be alleviated.

Another aspect of the invention also pertains to a method for improving the prognostic of an individual having a lesion in the CANS comprising the step of:
a) assessing the integrity of the CANS of said individual prior to the administration of a treatment;
b) administering to the said individual azasetron or an analogue thereof;
c) assessing the integrity of the CANS upon administration of the treatment as in step b);
   wherein the absence of deterioration of the integrity of the CANS at step c) is indicative of an improvement of the prognostic.

Within the scope of the invention, “improving the prognostic” refers to an increase or a cessation of a decrease of the quality of life following the occurrence of a trauma resulting in a lesion in the CANS.

It is also understood that the prevention uses and methods provided by the instant invention may be applied to an individual being at risk for having and/or developing a lesion in the CANS.

In some embodiments, an individual being at risk for having and/or developing a lesion in the CANS may non-limitationally be selected from an individual practicing an activity with a chance of collision, e.g., rugby player, hockey player; an individual belonging to a military force; an individual undergoing a brain surgery.

Methods for assessing the integrity of the central auditory system are well known from the state-of-the-art, such as, e.g., an imaging method, an electrophysiologic method and/or by a psychoacoustic method. Illustratively, one may refer to the middle latency responses (MLR), the auditory brainstem responses (ABR), and cortical auditory evoked potentials.

### BRIEF DESCRIPTION OF THE DRAWINGS

FIG. 1 is a graph showing the hearing threshold shift of placebo versus (R)-azasetron besylate of young adult NMRI mice having undergone an acoustic trauma and treated for 14 days with placebo (curve 1) or 26.4 mg/kg (R)-azasetron besylate (curve 2) peroral.

FIG. 2 is a graph (cochleogram) showing a number of lost outer hair cells (OHC) with respect to the distance from the cochlear apex in NMRI mice treated with placebo (plain circles) or (R)-azasetron besylate (plain triangles) as in FIG. 1.

FIG. 3 is a photograph showing a hematoxylin and eosin (H&E) staining of ventral cochlear nucleus (VCN) slices obtained from NMRI mice treated as in FIG. 1.

FIG. 4 is a histogram showing the number of normalized cell densities per grid, after 14 days of treatment with either (R)-azasetron besylate initiated at immediately or 24/48/72 hours after acoustic trauma or placebo, calculated from the VCN slices obtained in FIG. 3. Histogram bar 1: placebo. Histogram bars 2-5: (R)-azasetron besylate.

FIG. 5 is a histogram showing the number of normalized cell densities per grid from sample of the inferior colliculus (IC) from mice treated as described in the legend of

FIG. 4. Histogram bar 1: placebo. Histogram bars 2-5: (R)-azasetron besylate.

### EXAMPLES

The present invention is further illustrated by the following examples.

### 1—Materials and Methods

### In Vivo Treatment And Audiometry (12 Months)

12 animals per group (young adult Naval Medical Research Institute (NMRI) mice), 96 animals in total.

day 0:

Treatment groups 1-4 +Placebo groups 1-4:

Frequency specific ABR-thresholds at 4; 8; 12; 16; 20; 24; 28; 32 kHz under anesthesia (Ketamine/Xylazine). At least 3 measurements per frequency were performed.

day 1:

### Treatment and Placebo Groups

Noise application was performed for 3 h under anesthesia, using a broad band noise 4-20 kHz, 115 dB SPL, free-field.

### Treatment and Placebo Groups

Post-traumatic (0; 24 h; 48 h or 72 h after noise exposure) initiation of oral administration of 26.4 mg/kg (R)-azasetron besylate or of the related placebo formulation, respectively, was performed once daily. Treatments were continued daily for 14 days.
Treatment (T) group 1 +Placebo (P) group 1: Treatment from d1-d14
Treatment (T) group 2 +Placebo (P) group 2: Treatment from d2-d15
Treatment (T) group 3 +Placebo (P) group 3: Treatment from d3-d16
Treatment (T) group 4 +Placebo (P) group 4: Treatment from d4-d17

day 0-18:

Animals were kept in housings in a quiet surrounding at normal light/dark regime (12h/12h) and with free access to food and water. Cages were equipped with enrichment. Animals were kept in small groups.

days 15 (T/P groups 1), 16 (T/P groups 2), 17 (T/P groups 3) and 18 (T/P groups 4):

Treatment groups 1-4 +Placebo groups 1-4:

Frequency specific ABR-thresholds at 4; 8; 12; 16; 20; 24; 28; 32 kHz under anesthesia (Ketamine/Xylazine). At least 3 measurements per frequency were performed.

### Histology (3 Months)

Six animals per group (48 in total) have been perfused via the left hearth chamber with a fixative solution (paraformaldehyde (4%)) on day 15.

Cochleae and brains were prepared (cutting, staining), as disclosed in Gröschel et al. (2010). Cochlear hair cell loss and neuronal cell density in the ventral cochlear nucleus (VCN), inferior colliculus (IC) have been quantified.

### 2—Results

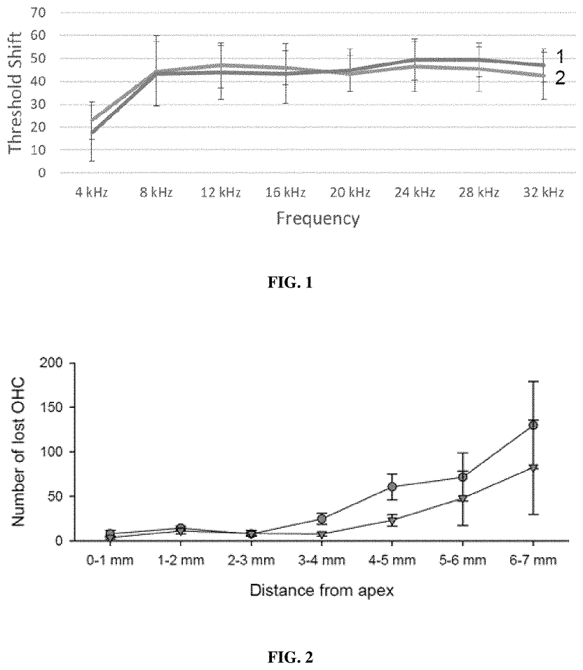

As seen in FIG. 1, the administration of (R)-azasetron besylate to rats subjected to a noise trauma does not significantly affect the threshold shift of the auditory brainstem response, as compared to the administration of placebo.

However, FIG. 2 shows that (R)-azasetron besylate affects the loss of outer hair cells (OHC) in the cochlea at growing distances from the apex.

The further hypothesis that a (R)-azasetron besylate mediated treatment of a noise trauma may likely benefit the auditory structures that are downstream from the inner ear (cochlea) was then assessed.

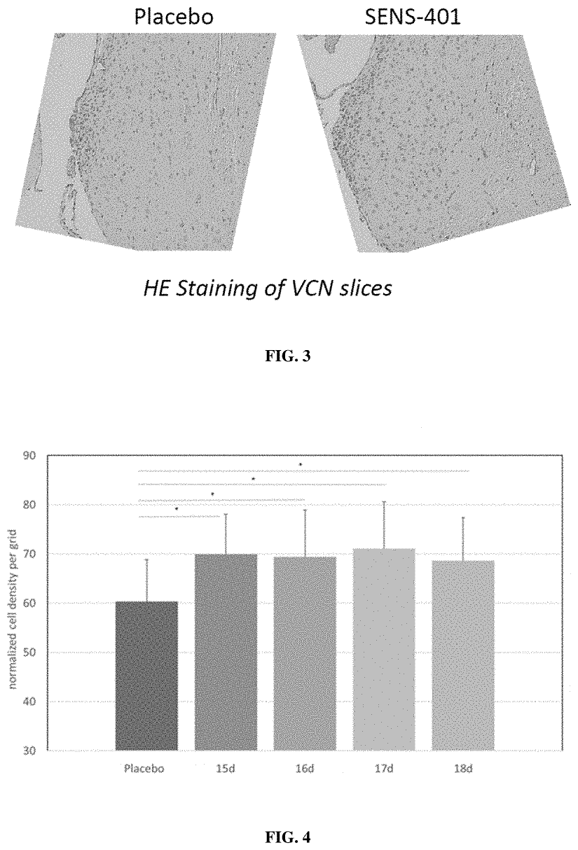

In order to confirm this theory, slices of ventral cochlear nucleus (VCN) were prepared for histological analysis (FIG. 3) and the total (neuronal) cells were counted (FIG. 4). FIGS. 3 and 4 show that the treatment with (R)-azasetron besylate reduces the destruction of neuronal cells in the VCN after a noise trauma.

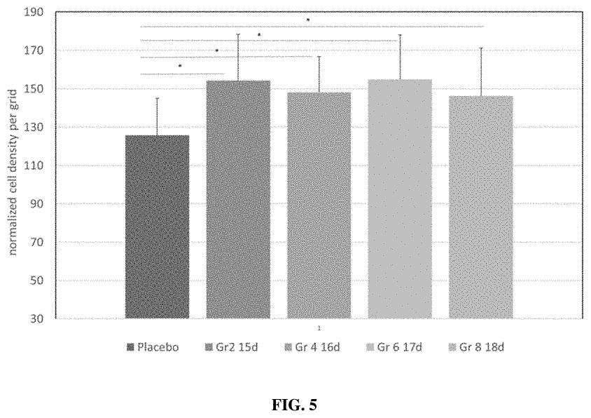

FIG. 5 similarly shows that (R)-azasetron besylate treatment of the mice subjected to an acoustic trauma also reduces the destruction of neuronal cells in a second structure of the CANS, the IC, as compared to the placebo treatment, irrespective of whether the treatment is initiated 0 h, 24 h, 48 h or 72 h after the acoustic trauma.

Altogether, these data indicate that a treatment of individuals having experienced a noise trauma with (R)-azasetron besylate will benefit the structures of the central auditory nervous system.

### 3—Discussion

To date, there are no models for studying a trauma in the CANS exclusively. Current models to achieve better knowledge on how the brainstem cope with trauma in the CANS are based upon an acoustic trauma. The acoustic trauma often results in lesions in the peripheral system, such as, e.g., the inner ear. Lesions of the peripheral system may be accountable for cochlear hair cell loss.

Nevertheless, under some controllable conditions, acoustic trauma may also be accountable for lesion in the CANS, in particular in the brainstem, the diencephalon, the mesencephalon, the auditory cortices.

Experimental data provided herein show that neurons within the ventral cochlear nucleus (VCN) and the inferior colliculus (IC) in the CANS are affected by an acoustic trauma, since cellular loss may be observed, most probably because of an apoptotic process. Administration of azasetron upon the onset of lesions allows to significantly reduce the loss of neurons in the brainstem.

Altogether, these data show that applying an acoustic trauma is a suitable model for studying lesion occurring in the CANS and that azasetron may promote inhibition or reduction of the apoptotic mechanism that undergoes upon the occurrence of the lesion.

1-15. (canceled)

16. A method for treating and/or preventing a lesion in the central auditory nervous system (CANS) in an individual in need thereof, said method comprising administering to said individual a therapeutically effective amount of azasetron, or an analogue thereof.

17. The method according to claim 16, wherein azasetron, or the analogue thereof, is in the form of a pharmaceutically acceptable salt and/or solvate thereof.

18. The method according to claim 16, wherein azasetron is (R)-azasetron, (S)-azasetron, or a mixture thereof.

19. The method according to claim 16, wherein azasetron is (R)-azasetron or a pharmaceutically acceptable salt and/or solvate thereof.

20. The method according to claim 19, wherein the pharmaceutically acceptable salt of (R)-azasetron is selected from the group consisting of (R)-azasetron besylate, (R)-azasetron malate, and (R)-azasetron hydrochloride.

21. The method according to claim 16, wherein azasetron, or the analogue thereof, is administered at a dose ranging from about 0.01 mg/kg of body weight to about 100 mg/kg of body weight.

22. The method according to claim 16, wherein azasetron, or the analogue thereof, is administered systemically.

23. The method according to claim 16, wherein azasetron, or the analogue thereof, is administered transdermally.

24. The method according to claim 16, wherein azasetron, or the analogue thereof, is administered orally.

25. The method according to claim 16, wherein said lesion in the CANS is a lesion localized in the cochlear nucleus, the superior olive, the trapezoid body, the lateral lemniscus, the inferior colliculus, the medial geniculate body, and/or the auditory cortices.

26. The method according to claim 16, wherein said lesion in the CANS is a lesion observed in a disorder selected from the group consisting of a central auditory processing disorder, a stroke of the central auditory pathway, a seizure, a brain trauma, a bacterial or viral infection, a demyelinating disorder, a neurodegenerative disorder, a neoplasm, a brain malformation, learning disabilities, dyslexia, autism, depression, alcoholism, anorexia, schizophrenia, epilepsy, an infantile mental retardation, an attention deficit disorder, and aged-related degeneration.

27. The method according to claim 16, wherein said central auditory processing disorder is an auditory processing deficit and/or a loss of speech recognition.

28. The method according to claim 16, wherein azasetron, or the analogue thereof, is administered in combination with at least one pharmaceutically acceptable excipient in a pharmaceutical composition.

29. A method for preventing and/or inhibiting and/or reducing the loss of central auditory neuronal cells in an individual in need thereof, comprising administering to said individual a therapeutically effective amount of azasetron or an analogue thereof.

30. The method according to claim 29, wherein azasetron, or the analogue thereof, is in the form of a pharmaceutically acceptable salt and/or solvate thereof.

31. The method according to claim 29, wherein azasetron is (R)-azasetron, (S)-azasetron, or a mixture thereof.

32. The method according to claim 29, wherein azasetron is (R)-azasetron or a pharmaceutically acceptable salt and/or solvate thereof.

33. The method according to claim 32, wherein the pharmaceutically acceptable salt of (R)-azasetron is selected from the group consisting of (R)-azasetron besylate, (R)-azasetron malate, and (R)-azasetron hydrochloride.

34. The method according to claim 29, wherein said individual is susceptible to undergo or have undergone an acoustic trauma.
